(19) United States
(12) Patent Application Publication
(10) Pub. No.: US 2020/0375691 A1
(43) Pub. Date: Dec. 3, 2020

(54) PERACETIC ACID VAPOR STERILIZATION SYSTEM

(71) Applicant: Medivators Inc., Plymouth, MN (US)

(72) Inventors: Kristopher Murphy, New Brighton, MN (US); Huyen Bui, Brooklyn Park, MN (US); John Matta, Shoreview, MN (US); Tuan Nguyen, Chaska, MN (US)

(21) Appl. No.: 16/967,466

(22) PCT Filed: Feb. 11, 2019
(86) PCT No.: PCT/US19/17425

§ 371 Date: Aug. 5, 2020

Related U.S. Application Data
(60) Provisional application No. 62/629,306, filed on Feb. 12, 2018.
(60) Provisional application No. 62/771,240, filed on Nov. 26, 2018.

(51) Int. Cl. A61B 90/70; A61L 2/07; A61L 2/18; A61L 2/26

(52) U.S. Cl. CPC A61B 90/70; A61L 2/07; A61L 2/186; A61L 2101/36; A61B 2090/701; A61L 2202/122; A61L 2202/14; A61L 2/26

(57) ABSTRACT

Systems and methods are disclosed for forming a vaporized peracetic acid, with sterilization taking place at ambient temperature. In particular, the disclosed peracetic bubbler technology may achieve sustained peracid acid concentrations. This bubbler and vaporization technology requires no further mechanical assistance, such as pumps, to move peracetic acid vapor into the interior components of an endoscope or any other medical device or medical appliance.

### PRIORITY CLAIM

This application claims priority to and benefit of U.S. Provisional Application with Ser. No. 62/629,306, filed on Feb. 12, 2018, and U.S. Provisional Application with Ser. No. 62/771,240, filed on Nov. 26, 2018, which are herein incorporated by reference in their entireties.

### BACKGROUND

An effective, commonly used method for sterilizing surgical supplies and drugs is by steam under pressure. However, sterilization at high temperatures adversely affect many surgical instruments and supplies thereby limiting products that can be steam sterilized and adding expense to the sterilization process.

Ethylene oxide has been promoted as an effective sterilization method offering lower temperatures. But ethylene oxide sterilization is not without its disadvantages. For example, ethylene oxide leaves behind chemical residuals that can create quality, safety, and regulatory concerns.

### SUMMARY

Disclosed are a method, and a vaporization and sterilization system. The method and system allow for using vaporized peracetic acid (PAA) for sterilizing at room or ambient temperature. Ambient temperature eliminates or reduces the potential for thermal degradation of associated equipment thereby expanding the materials and products that can be sterilized. The disclosed method and system also provides effective microbial sterilization, reduces the residuals, and breaks down PAA into non-toxic oxygen, water, and carbon dioxide.

In one embodiment, the invention relates to a PAA sterilizing system comprising:
a PAA vapor generator configured to vaporize a liquid PAA mixture;
a sterilization chamber in fluid communication with the vapor generator and configured to receive vaporized PAA, the sterilization chamber at a temperature in the range from 18° C. to 35° C.; and
a clean-up line in communication with the sterilization chamber capable of removing the vaporized PAA.

In another embodiment, the invention relates to a PAA sterilizing system comprising:
a source of pressurized gas;
a PAA vapor generator configured to receive the pressurized gas and vaporize the PAA;
a sterilization chamber in fluid communication with the vapor generator and configured to receive the vaporized PAA, the sterilization chamber at a temperature in the range from 18° C. to 35° C.;
a vapor recirculation system that recirculates the vaporized PAA in the sterilization chamber; and
a clean-up line in communication with the sterilization chamber capable of removing the vaporized PAA after recirculation.

In another embodiment, the invention relates a method of sterilizing an endoscope comprising
bubbling a gas through an aqueous PAA solution at a temperature less than about 60° C. to generate peracetic vapor,
   delivering the PAA vapor to a sterilization chamber containing an endoscope needing to be sterilized, wherein the sterilization chamber is at a temperature of about 18° C. to 35° C.;
   contacting the outer surfaces and inner channels of the endoscope with the PAA vapor,
   recycling the PAA vapor into the sterilization chamber until the endoscope is sterilized; and
removing the PAA from the sterilization chamber by applying a vacuum source to the sterilization chamber to provide the sterilized endoscope.

In still another embodiment, the invention relates to a method of sterilizing a medical device comprising:
bubbling a gas through an aqueous PAA solution at a temperature less than about 60° C. to generate PAA vapor;
delivering the PAA vapor to a sterilization chamber containing a medical device needing to be sterilized, wherein the sterilization chamber is at a temperature of about 18° C. to 35° C.;
contacting the outer surfaces and inner surfaces of the medical device with the PAA vapor;
recycling the PAA vapor in the sterilization chamber until the medical device is sterilized; and
   removing the PAA from the sterilization chamber by applying a vacuum source to the sterilization chamber to provide the sterilized medical device.

In a related embodiment, there is provided a PAA sterilization system that includes a PAA bubbler device configured to contain an aqueous PAA solution, the PAA bubbler device having an elongate container configuration with an elongated inlet tube located longitudinally therein with a distal portion of the inlet tube disposed adjacent a floor of the elongate container and a proximal portion of the inlet tube protruding beyond a top of the elongate container and open to a source of gas, the PAA bubbler device having an outlet tube disposed through the top of the elongate container with a distal portion of the outlet tube located within the elongate container in an open space above a maximum level for the PAA solution and with a proximal portion of the outlet tube providing an outflow path for a PAA gas. The system further includes a vacuum source and a vacuum chamber coupled to the PAA bubbler device. The PAA bubbler device contains a maximum level of the PAA solution that is adapted to be partially enclosed in the vacuum chamber with the proximal portion of the inlet tube and the proximal portion of the outlet tube extending outside of the vacuum chamber, an outflow of an PAA vaporized gas through the outlet tube being controlled by the vacuum source at a PAA gas flow rate defined by a height and volume of the elongate container and a length of the elongated inlet tube. The bubbler technology disclosed herein may achieve sustained PAA concentrations greater than those seen to date using, for example, pressurized gas or air assisted vaporization. This technology requires no further mechanical assistance, such as pumps, to move PAA vapor into, for example, the elevator wire channel of a duodenoscope (or any other endoscope small lumen) or any other medical device or medical appliance.

In a related embodiment, a PAA apparatus is provided that includes a PAA bubbler vessel configured to contain an aqueous PAA solution, the PAA bubbler vessel having an elongate container configuration with an elongated inlet tube located longitudinally therein with a distal portion of the inlet tube disposed adjacent a floor of the elongate container and a proximal portion of the inlet tube protruding from a top of the elongate container and open to a source of gas, the PAA bubbler device having an outlet tube disposed through the top with a distal portion of the outlet tube located within the elongate container in an open space above a maximum level of the PAA solution and with a proximal portion of the outlet tube providing an outflow path for a PAA gas. The PAA bubbler vessel contains the maximum level of the PAA solution and is partially subjected to a vacuum environment with the proximal portion of the inlet tube and the proximal portion of the outlet tube extending outside of the vacuum environment. An outflow of a PAA gas through the outlet tube is controlled by the vacuum environment at a flow rate defined by a height and volume of the elongate container and a length of the inlet tube.

In yet another related embodiment, a method of sterilizing a medical appliance is provided comprising the steps of providing a PAA bubbler device containing an aqueous PAA solution, the PAA bubbler device having an elongate container configuration with an elongated inlet tube located longitudinally therein with a distal portion of the inlet tube disposed adjacent a floor of the elongate container and a proximal portion of the inlet tube protruding from a top of the elongate container and open to a source of gas, the PAA bubbler device having an outlet tube disposed through the top with a distal portion of the outlet tube located within the elongate container in an open space above a maximum level of the PAA solution and with a proximal portion of the outlet tube providing an outflow path for a PAA gas. The method further includes the step of subjecting the PAA bubbler device containing the PAA solution to a vacuum environment such that the proximal portion of the inlet tube and the proximal portion of the outlet tube extend outside of the vacuum environment. The method also includes the step of bubbling the source gas through the PAA solution from the inlet tube initiated by the vacuum environment and generating an outflow of a PAA vapor gas through the outlet tube, wherein the elongate container is of a defined height and volume and the elongated inlet tube is of a defined length to facilitate an inflow of a gas through the inlet tube at a defined flow rate. In a related embodiment, the step of subjecting the PAA bubbler device includes the step of providing a vacuum of to a range of about 0.1 torr to about 500 torr, alternatively about 0.1 to about 100 torr, and subjecting the endoscope to a PAA vapor gas flow for a time period of about 10-60 minutes. In one embodiment, the medical appliance is an endoscope and the method further includes the step of connecting the outlet tube of the PAA bubbler device to an inlet of the endoscope for sterilizing internal channels or lumens such as an elevator wire channel.

### BRIEF DESCRIPTION OF THE DRAWINGS

FIG. 1 illustrates an exemplary vaporization and sterilization system.

FIG. 2 is a schematic diagram of an endoscope illustrating interior channels and related valves and connectors.

FIG. 3 illustrates an exemplary hookup scheme to an endoscope.

FIG. 4 illustrates an exemplary hookup scheme to an endoscope.

FIG. 5. illustrates an exemplary enclosure for sterilization.

FIGS. 6A-6C illustrate another exemplary vaporization vessel for use in an improved vaporization system.

FIG. 7 illustrates a block diagram of the improved vaporization system.

FIG. 8 illustrates a graph of a PAA signal for various methods of PAA introduction.

FIGS. 9A-9D illustrate exemplary vaporization vessels of differing sizes and configurations, respectively, for use in a vaporization system illustrated in a block diagram.

FIG. 10 illustrates an embodiment of a PAA vaporization bubbler having two vessels in a vaporization system.

FIGS. 11A-1C illustrate graphs of a bubbler flow rate, PAA signals for PAA introduction and relative humidity data from an oversized bubbler of FIG. 9B, respectively.

FIG. 12 illustrates a comparison of gas phase and solution phase structures.

FIG. 13 is a diagrammatic representation of gas phase to PAA energetics.

### DETAILED DESCRIPTION

The term “microorganism (s)” refers to any non-cellular or unicellular organisms. Microorganisms include prokaryotes such as bacteria (including cyanobacteria), and spores thereof; and eukaryotes such as algae, fungi, yeast. The term microorganism also encompasses viruses and virus particles.

The term “sterilization” or any grammatical equivalent thereof refers to a reduction or elimination of microorganisms.

The phrase “reduction of microorganisms” refers to at least a 50% reduction in a microbial population.

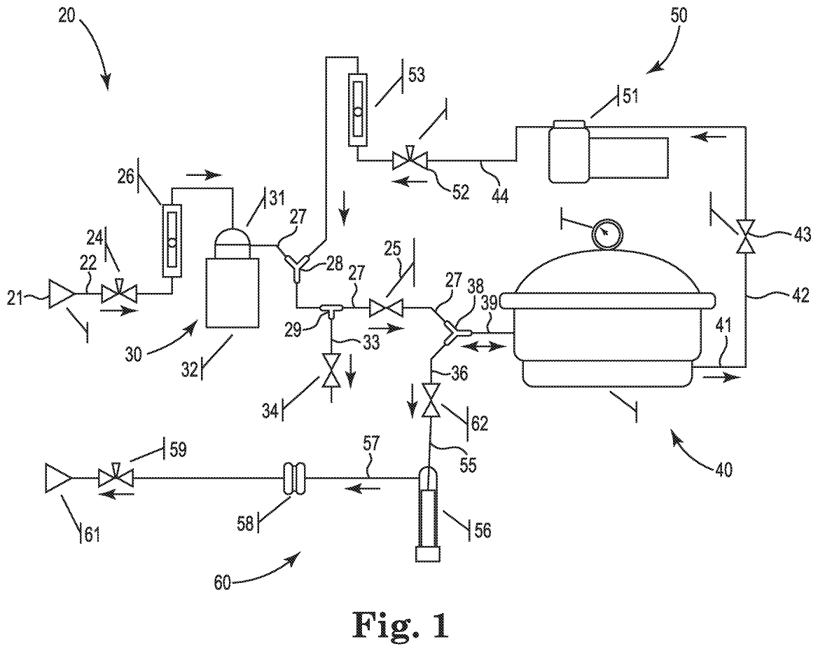

FIG. 1 illustrates an embodiment of PAA vapor system 20 for decontaminating or sterilizing equipment and objects with vaporized PAA. The system 20 includes vapor generator 30, sterilization chamber 40, recirculating pump 50 and clean-up or exhaust line 60. Vapor from the vapor generator 30 is supplied to chamber 40 via conduits 27 and 39. The vapor then exits sterilization chamber 40 and is recirculated within sterilization chamber 40 with recirculation pump 50 and associated conduits. The recirculated PAA vapor can be combined with fresh PAA vapor and delivered back to the sterilization chamber 40 with the objects to be sterilized. After the desired exposure time, the PAA vapors are withdrawn from sterilization chamber 40 under reduced pressure (e.g., by application of a vacuum) and through clean-up line 60, that includes a chemistry trap and particle filter to neutralize exhaust vapor.

The vapor generator 30 can be one of a sparger or bubbler 31, which includes a vessel or container containing an aqueous PAA solution, a bubbler connected to a source of pressurized gas 21 that is introduced into the vessel and the gas is bubbled into the solution to generate PAA vapor. The PAA vapor exits sparger 31 via conduit 27 into sterilization chamber 40. An air throttle valve 24 may be present between the source of gas and sparger 31 to control the air flow. The vapor generator 30 can also include a heating component 32 and a conduit having valve 28 connected from vapor generator 30 to sterilization chamber 40. The vapor generator can be made of any suitable material, such as glass, plastic, or metal, which is inert to the PAA source contained therein. Stainless steel is preferred in some embodiments.

Bubblers typically have a downtube inlet where gas is introduced into the container under the surface of the liquid chemical precursor where the gas bubbles up through the liquid solution, entraining the PAA as the gas surfaces the liquid as a bubble and exits the container or bubbler by an outlet set above the liquid level of the PAA as a PAA vapor. The gas may include air, argon, or nitrogen.

Between vapor generator 30 and sterilization chamber 40 is vapor/liquid separator 34, designed to remove or trap any liquid droplets present in the vapor and to prevent passage of liquid to the objects to be sterilized, such that the object is exposed to vapor only. A number of suitable techniques are available for removing liquids from vapor. The choice of a particular technique depends on the vaporization rate and the volume and flow rate of the gas. For example, a bent or angular conduit, or one containing one or more baffles may be suitable for small units, while a cyclone-type separator may be more appropriate for larger units. Illustrated in FIG. 1 is a moisture trap drain valve. In embodiments, the moisture trap and moisture trap drain valve are placed in line between the vapor generator and sterilization chamber.

Sterilization chamber 40, where the objects to be sterilized are placed, includes conduit 39 for introducing PAA vapor and an exit conduit 41 for exiting the PAA vapor and recirculating the PAA vapor. The vaporized PAA exiting via conduit 27 passes through a connector 28 and 38 to enter the chamber 40.

The sterilization chamber is configured to be substantially free of internal corners or edges, which can affect the sterilization process and circulation flow. Accordingly, the chamber can have a substantially ellipsoid, ovoid, or spheroid shape. The sterilization chamber can be formed of any suitable material such as metal (e.g. aluminum or stainless steel), ceramic or a composite, such as an outer metal structure for strength and an inner ceramic liner for heat insulation.

The process can begin by vaporizing the PAA. A gas source 21 through a conduit or supply line 22 bubbles gas through liquid PAA contained in vapor generator 30. The conduit 22 between the gas source and vapor generator can include valve 24 (e.g., air throttle valve) for adjusting gas flow rate. In one embodiment for a 30 cm bubbler vaporization vessel or container, the gas can be bubbled through the aqueous PAA contained in the vapor generator at a flow rate of about 1000 to about 5000 ml/minute, or 2000-5000 ml/minute. The vapor generator can also include a heat element, such as an electric heater having the capability of heating the liquid contents in the vapor generator from about 40° C. to about 60° C.

Sterilization of the objects to be sterilized begins by air being removed from the sterilization chamber with the aid of a hemostat 36 and 25. Sterilization under vacuum can shorten the sterilization time. The vaporized PAA is then supplied into sterilization chamber 40 and the sterilization chamber is maintained at ambient temperature (e.g., about 15° C. to 40° C.). In embodiments, the sterilization chamber is at about a temperature of about, 18° C. to 35° C., 20° C. to about 30° C., or 25° C. to 35° C.

The PAA vapor is continuously circulated through the sterilization chamber 40 and the objects to be sterilized for the desired time such that the object is sterilized. Circulation of the PAA vapor can occur via a vapor recirculation system 50, which includes a diaphragm pump 51, circulation throttle valve 52, circulation flow meter 53, circulation vacuum isolation hemostat 43 and conduits 42 and 44, that together that allow the PAA vapor to pass from the sterilization chamber 40 to the diaphragm pump 51 and back to the sterilization chamber 40 via the circulation flow meter 53.

The PAA vapor to the sterilization chamber 40 can be delivered at a rate of about 2000-12000 ml/minute. In some embodiments, the objects to be sterilized are exposed to the PAA vapor for about 15 to 60 minutes.

In some embodiments, vapor generator 30 provides PAA vapor to the sterilization chamber 40 at a PAA concentration of about 500-2500 ppm, a hydrogen peroxide concentration of less than about 100 ppm (in some cases the concentration is down to less than 1ppm) and an acid concentration of less than about 100 ppm.

When sterilization is complete, circulation throttle valve 52 and hemostat 25 (e.g. sparger vacuum isolation) are closed. The PAA vapor can be pumped out via the same conduit 39 that branches out through evacuation line 36 into a clean-up line 60. Clean-up line 60 can include a trap 56 and particle filter 58 to neutralize evacuated PAA vapor and to remove undesirable ingredients from the vapor or to decrease the concentration of such undesirable ingredients from the vapor. The clean-up line can further include a vacuum 61 and a vacuum throttle valve 59. In embodiments, the clean-up line includes a moisture trap and moisture trap drain valve. In other embodiments, the clean-up line is in communication with the sterilization chamber through hemostat 62.

Optionally, air can be used to purge the chamber after sterilization is completed. In some cases, a fan can be associated with the conduit 39, or a pump can be used to pull the vapor out of the chamber or a combination of both may be used. Any suitable system and method can be used to reduce or remove from the chamber the PAA vapor concentration as fast and as efficiently as possible.

Because the sterilization can be conducted at room temperature it expands the objects that can be sterilized using the vaporizer system and method. Such objects can include products and materials that include medical device, sensors and biologicals. Medical devices include endoscopes (e.g., arthroscope, cystoscope, nephroscope and the like).

When the system and method is used to sterilize endoscopes, the sterilization chamber can include connections that are capable of connecting an endoscope. Such connections will be referred to as endoscope hookup connectors. The endoscope hookup connectors allow exposure of the internal endoscope chancels, chambers, and lumens as well as the external endoscope.

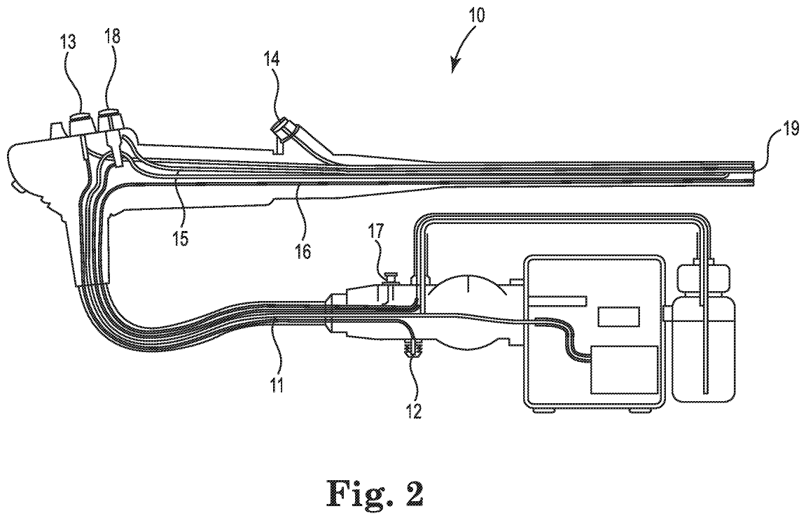

FIG. 2 schematically illustrates the interior channel architecture of an endoscope 10 as well as the related valves and access connectors of the endoscope that allow the PAA vapor to be added to the interior channels and lumens during sterilization. The biopsy/suction channel 11 may be accessed by or connected to vapor sterilant source through suction connector 12, suction valve port 13 or biopsy channel inlet 14. Water channel 15 and water-jet channel 16 may be accessed by or connected through water-jet connector 17 or air/water valve port 18. When an endoscope is sterilized using the vaporizer system and method, the endoscope can be connected to the sterilization system in different configurations depending on the endoscope.

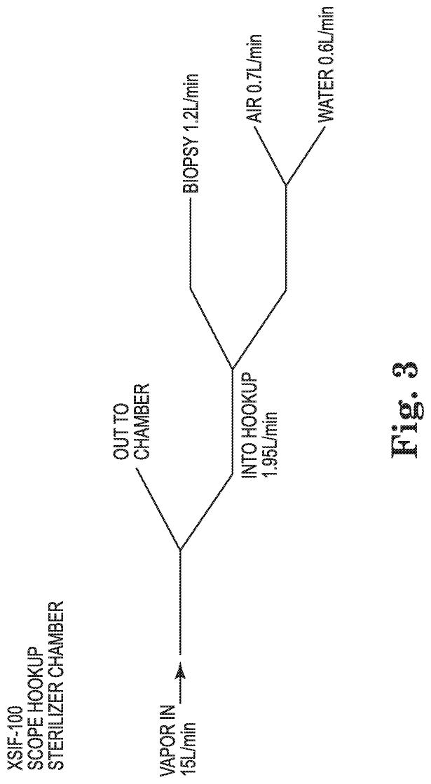

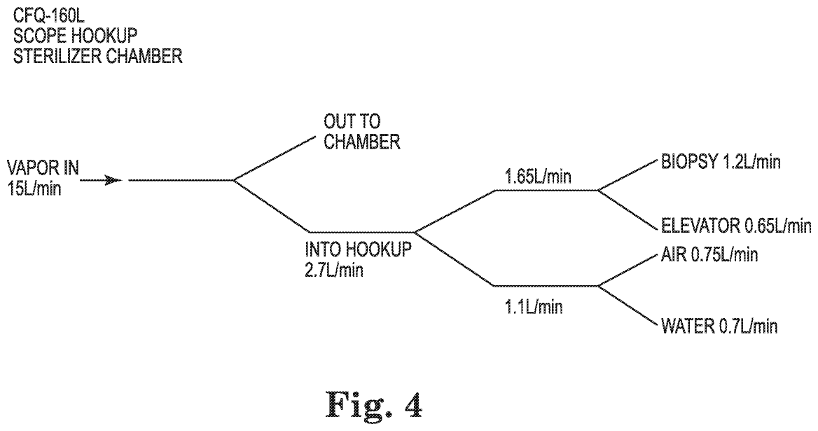

FIGS. 3 and 4 illustrate various schemes, SCHEME A and SCHEME B, respectively, by which the endoscope hookup connectors can be connected to an endoscope for sterilization.

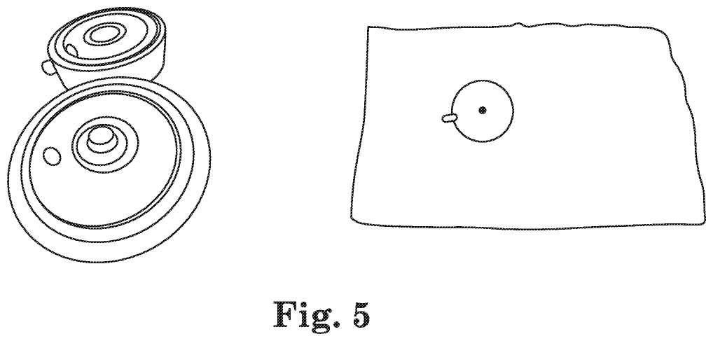

FIG. 5 illustrates a packaging material, pouch or bag 500 that may be used for sterilizing objects. The bag can include adaptors 501 and 502 to connect the bag to the disclosed system and be placed in the sterilization chamber. The advantage of using such a bag in the disclosed system is that is maintains a sterile environment for the object that is held in the bag after it has been removed from the sterilization chamber. An example of suitable packaging material can include Tyvek bags. Any material, pouch or bag that can withstand the sterilization chamber conditions and maintain the sterility of the object within may be used with the disclosed system.

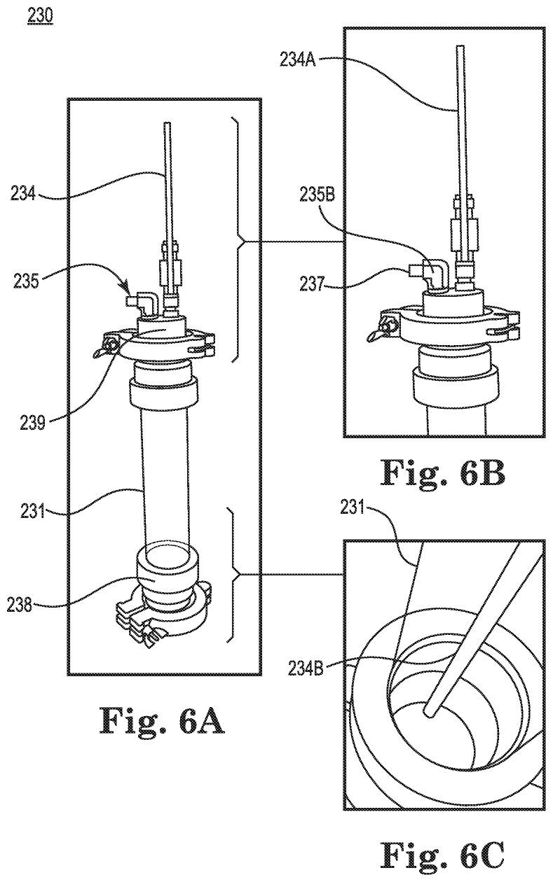

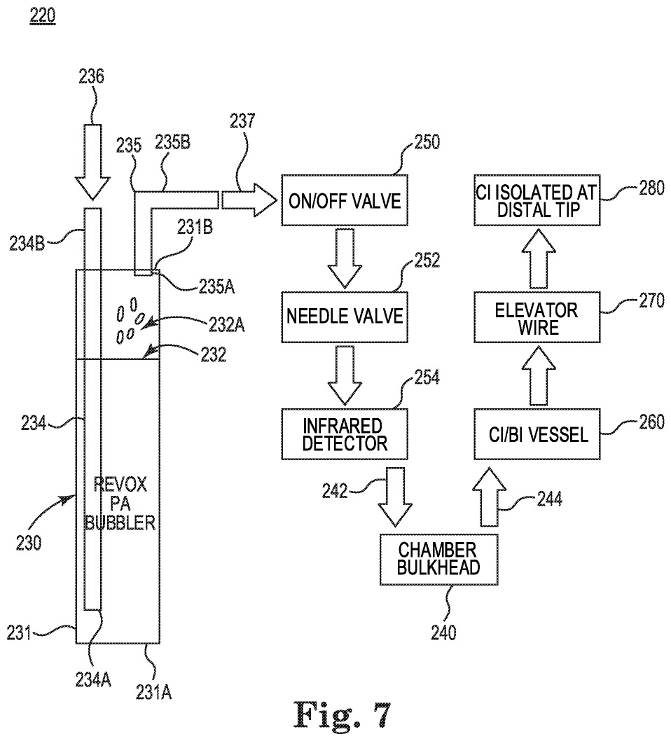

In other embodiments, FIGS. 6A-6C illustrate an alternative vaporization vessel 230 to be used in an embodiment of an alternate PAA vapor system 220 illustrated in FIG. 7. This PAA vapor system is useful for decontaminating or sterilizing equipment and objects with vaporized PAA. In this embodiment, system 220 includes vapor generator 230 having a bubbler vessel 231 with an inlet tube 234 and an outlet tube 235. The outlet tube 235 has a proximal end 235A disposed at a top end of vessel 231 and a distal end or outlet 235B. Outlet 235B is coupled to an on/off valve 250 which in turn is coupled to a needle valve 252. An output of needle valve 252 is coupled to an inlet 242 of a sterilization chamber or bulkhead 240 with an outlet 244 of bulkhead 240 being coupled to an endoscope 270 (not shown). In this embodiment, there is optionally included a vessel 260 that includes both chemical and biological indicators for PAA detection and measurement and an optional chemical indicator at a distal tip 280 of endoscope 270 to detect the existence of PAA in the outlet of vaporization system 220.

Again referring to FIGS. 6A-6C and 7, vapor generator 230 includes a sparger or bubbler vessel 231, which includes a vessel or container (in this embodiment a glass container or tube) containing an aqueous PAA solution 232, with bubbler vessel 231 configured to draw air 236 (or any other gas) through an elongate inlet tube 234 that is introduced into the vessel and the gas is bubbled into the solution to generate PAA vapor 232A. PAA vapor 232A exits bubbler 231 via conduit 235 through on/off valve 250 and needle valve 252 (to control vapor flow) and past infrared detector 254. PAA vapor 232A is then introduced into sterilization chamber 240. In a related embodiment, vapor generator 230 can also include a heating component (not shown) that is wrapped around the vessel prior to flowing into sterilization chamber 240. The vapor generator can be made of any suitable material, such as glass, plastic, or metal, which is inert to the PAA source contained therein. Glass and stainless steel, individually and combinations thereof, are preferred in some embodiments.

Although not shown, sterilization chamber 240, where the objects to be sterilized are placed, includes an inlet conduit 242 for introducing PAA vapor and an outlet conduit 244 for exiting the PAA vapor. The sterilization chamber may be configured to be substantially free of internal corners or edges, which can affect the sterilization process and circulation flow. Accordingly, the chamber can have a substantially ellipsoid, ovoid, or spheroid shape. The sterilization chamber can be formed of any suitable material such as metal (e.g. aluminum or stainless steel), ceramic or a composite, such as an outer metal structure for strength and an inner ceramic liner for heat insulation.

In operation and in one embodiment, PAA sterilization system 220 utilizes PAA bubbler device 230, which contains the aqueous PAA solution 232, configured from an elongate glass container 231 with an elongated inlet stainless steel tube that is located longitudinally within container 231 with a distal portion 234A of the inlet tube being disposed adjacent a floor 231A of elongate container 231 and a proximal portion 234B of the inlet tube protruding beyond a top 231B of the elongate container and open to a source of air or another gas. PAA bubbler device 230 has an outlet stainless steel tube 235 disposed through the top of the elongate container with a proximal portion 235A of the outlet tube located within the elongate container in an open space above a maximum level for PAA solution 232 and with a distal portion of outlet tube 235B providing an outflow path 237 for a PAA gas 232A. System 220 further includes a vacuum source (not shown) and a vacuum chamber (which in this example is chamber bulkhead 240) coupled to the PAA bubbler device 230. The PAA bubbler device contains a maximum level of the PAA solution that is partially enclosed in the vacuum chamber with the proximal portion of the inlet tube and the proximal portion of the outlet tube extending outside of the vacuum chamber. An outflow of the PAA vaporized gas that flows through outlet tube 235B is controlled by the vacuum source at a PAA gas flow rate defined by a height and volume of elongate container 231 and a length of the elongated inlet tube 234. In this embodiment, elongate container 231 is a 30 centimeter (cm) tube capable of containing 40 mL of REVOX PA sterilant and tube 234 is a 0.25 inch stainless steel tube that submerged into REVOX PA sterilant. Air 236 is drawn in through the inlet tube down to the bottom of the glass vessel and then bubbles up through the REVOX PA sterilant. The air vaporizes a portion of the PAA within the REVOX PA sterilant and then both air and PAA are then exhausted through outlet tube 235B.

In this embodiment, vapor system 220 was substantially modified to allow PAA vapor to flow from the bubbler 231 into an endoscope's elevator wire channel 270. To generate flow, a duodenoscope was placed in a poly tray (no terminal packaging was used) and the elevator wire channel of the endoscope was connected to the flow path. The vacuum chamber 240 was evacuated to 20 torr (±5 torr) so as to create enough pressure differential to flow PAA vapor 232A without any additional mechanical assistance, such as a pump. The flow rate was estimated to be about 150 ml per minute. Potential system leaks were assessed by closing the On/Off valve of the bubbler, isolating the chamber in manual control mode, and holding for 5 minutes. Chamber leak rate was determined to be less than 0.5 torr per five minutes. Depending on the application and the size of the bubbler vessel, PAA flow rate was to be controlled via the needle valve. In this illustrated embodiment, needle valve 252 was in its maximum flow position during all experiments, indicating needle valve 252 was not needed. Flow of PAA vapor to the distal tip of the scope was assured by affixing a PAA chemical indicator (CI) 280. Additional CIs were placed throughout the chamber to monitor for the presence of residual PAA presence, but these were optional and not critical to the operation of system 220.

Referring again to FIG. 7, in one embodiment, in system 220 using a 417 liter REVOX vessel the vacuum chamber evacuates to a range of about 15-25 torr and flows gas at a flow rate of about 40 liter/minute about 160 liter/minute through the elongate container or vessel as a function of the vacuum pressure and elongate container height and volume. System 220 draws gas through the proximal end 234B of the inlet tube 234, with the gases including anyone of air, argon, or nitrogen. System 220 provides PAA vapor to the sterilization chamber 240 at a PAA concentration of about 1 ppm, with a hydrogen peroxide concentration of less than about 0.1 ppm.

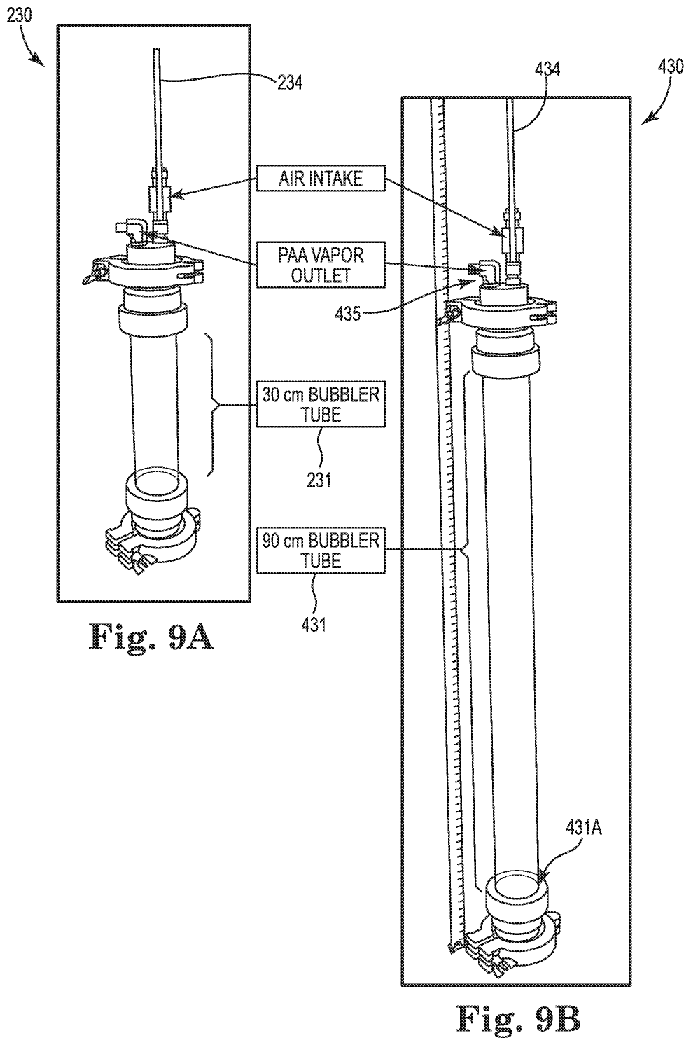

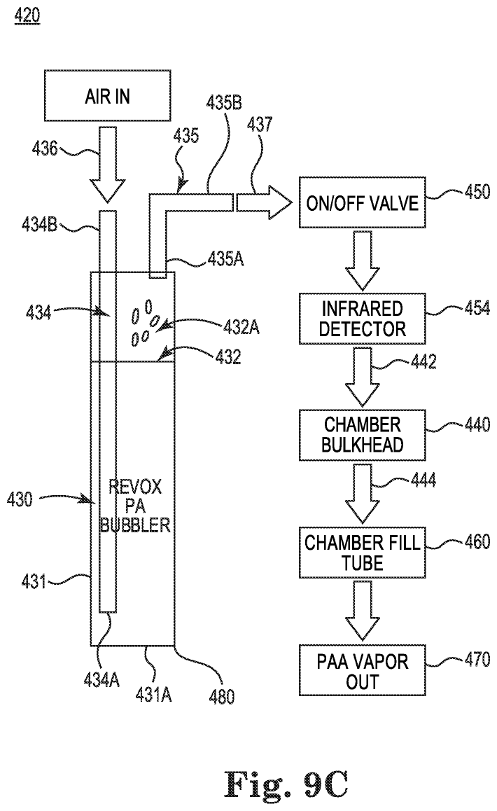

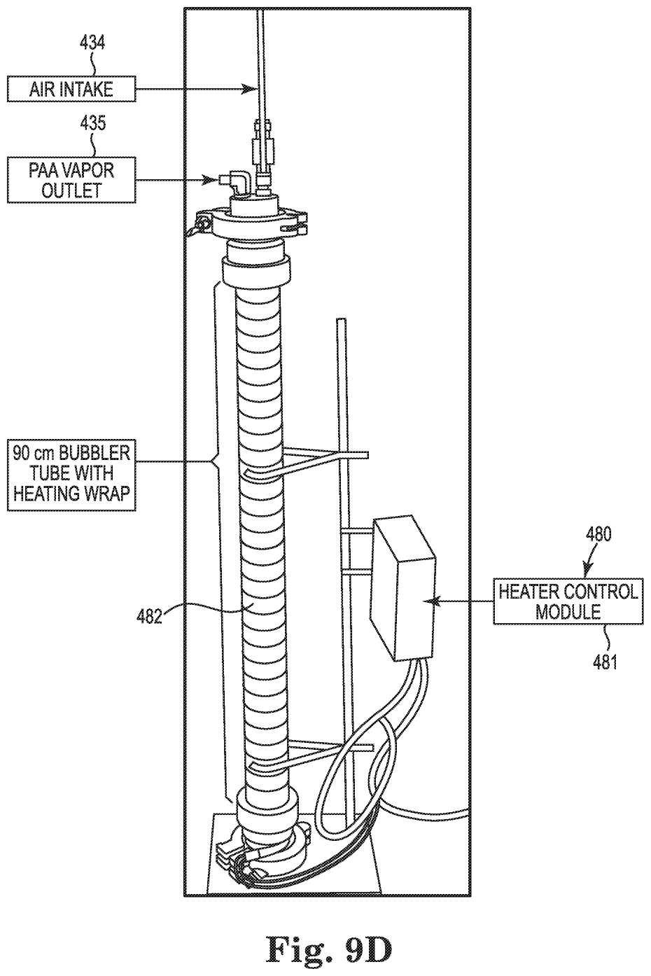

Referring now to FIGS. 9A-9D, there are illustrated exemplary vaporization vessels 230 and 430 of differing sizes and configurations, respectively, for use in an improved vaporization system 420. FIG. 9C illustrates a block diagram of the improved vaporization system 420 incorporating a vaporization vessel 430 illustrated in FIG. 9B. In particular, bubbler 230 described earlier is shown in comparison to another embodiment bubbler 430 (90 cm) which is larger and provides for improved PAA vaporization and can deliver near 70 LPM (liters per minute) of PAA vapor, making it adaptable to current 417-liter REVOX sterilization technology. Non-challenging objects or medical devices and appliances may achieve sterilization in less than 22 minutes using system 420 as described herein. Further, an advantage to this embodiment is this PAA vapor delivery mechanism does not leave significant residues of hydrogen peroxide or the non-volatile DEQUEST™ stabilizer which may be present in REVOX PA. Another discovery for improved delivery of PAA is that the PAA vapor requires a significant presence of water vapor to be efficacious. Heating of the bubbler vessel 430 advantageously, with a heating system 480 of a heating wrap 482 using and a heater control module 481, delivers the PAA vapor along with its needed water vapor to serve as an effective bactericidal agent.

In this example embodiment, improved vaporization vessel 430 is used in PAA vapor system 420 for decontaminating or sterilizing equipment and objects with vaporized PAA, In this example embodiment, system 420 includes vapor generator 430 having a bubbler vessel 431 with an inlet tube 434 therein and an outlet tube 435. The outlet tube 435 has a proximal end 435A disposed at a top end of vessel 431 and a distal end 435B. Outlet 435B is coupled to an on/off valve 450 which in turn is coupled to an infrared detector 454 with an outlet of infrared detector 454 being coupled to an inlet 442 of a sterilization chamber or bulkhead 440 with an outlet 444 of bulkhead 440 being coupled to an elevator wire of an endoscope (not shown) or another medical appliance. In this embodiment, there is a chamber fill tube or vessel 460 and then a vaporization system outlet 470. Referring to FIG. 9D, heating system 480 with heater module 481 and heating member or wrap 482 is included that envelops most of bubbler vessel 431 so as to heat up the PAA fluid and vapor before it exits vessel 431. This combination of heat and humidity dramatically improves sterilization outcomes when sterilizing medical devices and appliances, such as endoscopes.

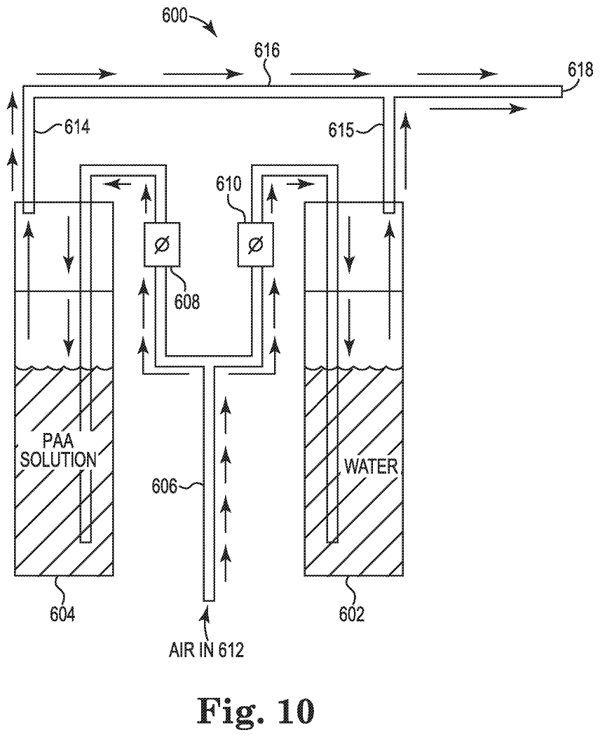

Referring again to FIGS. 9B and 9C, bubbler 431 is configured from a glass vessel or container (in this case a glass container or tube) containing an aqueous PAA solution 432, with bubbler 431 configured to draw air 436 (or any other gas) through elongate inlet tube 434 spanning the length of the glass vessel introducing the gas at the bottom 431A of the vessels holding the PAA fluid. Thereafter, the gas is bubbled into the solution to generate PAA vapor 432A which exits bubbler 431 via conduit 435 through on/off valve 450 and past infrared detector 454. Graphs of the PAA levels will be discussed in more detail in connection with FIGS. 10A-10C. PAA vapor 432A is then introduced into sterilization chamber 440. The vapor generator can be made of any suitable material, such as glass, plastic, or metal, which is inert to the PAA source contained therein. Glass and stainless steel, individually and combinations thereof, are preferred in some embodiments.

For a 417 liter chamber the flow rates were about 40 liter/minute to about 160 liter/minute. The vapor generator can also include a heat element, such as an electric heater, having the capability of heating the liquid contents in the vapor generator from about 40° C. to about 80° C.

In another embodiment, FIG. 10 illustrates an alternate PAA vapor system 600. This two vessel system includes a heated vessel or container 602 containing heated water and another vessel or container 604 containing an aqueous PAA solution. The water is heated to a temperature in the range of about 40° C. to about 80° C. Inlet tube 606 includes two flow control valves 608 and 610, respectively, which control a flow of compress gas from a compressed gas source 612 through inlet tube 606. The compressed gas flows into the heated water vessel 602 and aqueous PAA vessel 604 through a segment of inlet tube 606 submerged in the two liquids to generate water vapor and PAA vapor. These vapors exit the heated water vessel and aqueous gas vessel through exit tubes 614 and 615, respectively. The water and PAA vapors are mixed in exit tube 616 to provide a source of humidified PAA vapor that leaves the PAA vapor system 600 through the humidified PAA vapor outlet 618.

A number of advantages are realized by the disclosed method and system, particularly operating under ambient temperatures. Vaporization of the PAA is facilitated and can be achieved quickly and conveniently at predetermined temperatures, e.g., below 60° C., and below 50° C. This is of particular advantage when vaporizing PAA, which typically decomposes at temperatures below its boiling points at atmospheric pressure. Also, at lower or ambient temperature, PAA decomposes more slowly and is available longer to carry out sterilization. Since vaporization and sterilization can be accomplished at ambient temperatures the useful life of the reactive PAA molecules will be increased thereby increasing their effectiveness in the sterilization process. Further, ambient temperatures and pressures decrease the opportunity for undesirable side reactions and eliminate the hazards normally associated with the use of peroxides. The PAA decomposes to non-toxic oxygen, water and carbon dioxide.

While the system and method was described primarily with PAA, other suitable acids can include peracids such as saturated and unsaturated peralkanoic acids including peralkanoic acids having from 1 to 6 carbon atoms and halogenated derivatives thereof. Examples of suitable peracids include known PAA, halogenated PAAs, performic acid, perpropionic acid, halogenated perpropionic acids, perbutanoic acid and its halogen derivatives, perisovaleric acid and its halogen derivatives, percapronic acid and its halogen derivatives, percrotonic acid, monopersuccinic acid, monoperglutaric acid, and perbenzoic acid, for example. The halogenated peracids contain one or more chloro, bromo, iodo or fluoro groups. The preferred peracids are sufficiently volatile to form a vapor at temperatures less than 60° C. The peracid can be vaporized from a solution. A suitable PAA used in an embodiment of the invention is MINNCARE™ Cold Sterilant available from Mar Cor Purification, Inc., Cantel Medical Company, Plymouth, Minn.

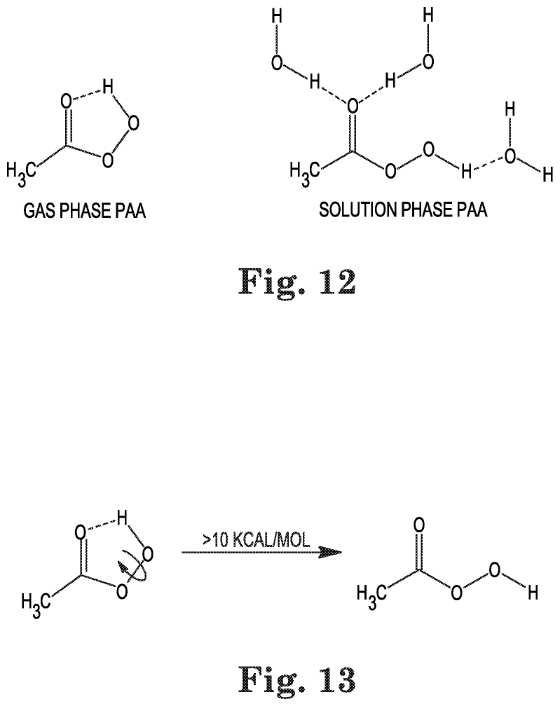

Referring to FIG. 12, there is illustrated a comparison of gas phase and solution phase structures. It is theorized that the structure of anhydrous PAA in the gas phase (in the absence of water) is a 5-membered cyclic structure created with an intramolecular hydrogen bond between terminal hydrogen to the carbonyl oxygen of PAA. Whereas, the solution phase structure is likely to be an open confirmation stabilized by numerous intermolecular hydrogen bonds to the available bulk water. Using a simple MM2 calculation and the dihedral drivers function in CambridgeSoft's Chem3D software, it is possible to roughly estimate the energetic cost of rotating the —O—O— bond of gas phase PAA. These calculations show PAA needs about 10 kilocalories per mol (kcal/mol) to achieve full bond rotation at the —O—O— bond. As a point of reference, a 3-4 kcal/mol difference would roughly equate to a 99:1 ratio of cyclic to open PAA structures. A 10 kcal/mol difference means far greater than 99% of the PAA vapor in a low humidity environment will be in the less active cyclic form.

Referring to FIG. 13, there is illustrated a diagrammatic representation of gas phase to PAA energetics. The cyclic, gas phase form of PAA allows the molecule to distribute electron density over a larger region than its linear form. This may have the effect of lowering the reactivity of gas phase PAA to the point where it is far less lethal to microorganisms. This is illustrated within the literature and has been confirmed with the non-heated bubbler experiments. In a high humidity gas phase environment, PAA vapor would take on a structure similar to the solution phase PAA structure shown in the Figure. This open structure of PAA does not allow for electron density to be distributed throughout the molecule and dramatically increases the reactivity of PAA. Further work is needed to confirm the electronic characteristics of PAA in the gas phase and determine exactly how much water needs to be present to activate PAA vapor.

Bubbler technology can be easily adapted and used on 417-liter REVOX sterilization chambers. It is scalable to chamber sizes smaller than 417-liters and potentially scalable to sizes greater than 417-liters. Bubbler 430 does not deliver significant amounts of hydrogen peroxide and should volatilize nearly zero DEQUEST stabilizer. This would allow comparatively fewer ventilation cycles as compared to air assisted atomization systems. PAA vapor generated by bubbler 430 and system 420 achieve substantial bactericidal activity when the PAA vapor is in the presence of water vapor as well. The ratio of water to PAA is estimated to be at least 10 to 1 ratio of water to PAA. For the PAA Bubbler technology, the measure of relative humidity can be used to determine when PAA vapor is efficacious. Data suggests this to be greater than or equal to 80% relative humidity. PAA vapor in the absence of water vapor likely exists in a cyclic conformation. This confirmation is less reactive, which causes dry PAA vapor to be less bactericidal. Non-heated Bubbler experiments did not deliver enough water vapor to activate the PAA in a significant manner and with little to no bacterial kill being observed. On the other hand, Heated Bubbler experiments deliver significant amounts of water vapor along with the PAA vapor allowing for sporicidal activity and rapid sterilization.

Since the sterilization can be conducted at room temperature it expands the objects that can be sterilized using the vaporizer system and method. Such objects can include products and materials that include medical device, sensors and biologicals. Medical devices include endoscopes (e.g., arthroscope, cystoscope, nephroscope, duodenoscope, and the like). When the system and method is used to sterilize endoscopes, the sterilization chamber can include connections that are capable of connecting an endoscope. Such connections will be referred to as endoscope hookup connectors. The endoscope hookup connectors allow exposure of the internal endoscope chancels, chambers, and lumens as well as the external endoscope.

The following patents and publications are incorporated by reference in their entireties: U.S. Pat. Nos. 8,821,807; 9,017,607; 9,402,929; US Publications 2016/0346416 and 2015/0202339.

### EXAMPLES

Embodiments of the present invention are further defined in the following non-limiting Examples. It should be understood that these Examples, while indicating certain embodiments of the invention, are given by way of illustration only. From the above discussion and these Examples, one skilled in the art can ascertain the essential characteristics of this invention, and without departing from the spirit and scope thereof, can make various changes and modifications of the embodiments of the invention to adapt it to various usages and conditions. Thus, various modifications of the embodiments of the invention, in addition to those shown and described herein, will be apparent to those skilled in the art from the foregoing description. Such modifications are also intended to fall within the scope of the appended claims.

### Example 1

A one liter glass bubbler as the vapor system was used to test three different flexible endoscopes to determine the efficacy of the system against microorganisms as a function of size and architecture of endoscope.

The three endoscope types were chosen to meet the three primary conditions of flexible endoscopes:
Long endoscope: XSIF-100
Complex channel system scope: CFQ-160L
Basic/small scope: BF-P40 and BF-P30

Each scope was inoculated on both the exterior and interior of the lumens with Geobacillus stearothermophilus suspended in serum or saline solution at a target concentration of microorganisms of 6-7 log for each run. The scope was then sealed inside a double Tyvek bag with a hookup adaptor. Each flexible endoscope was evaluated for a total of 22 inoculated surfaces to achieve a greater than 95% confidence level.

The test conditions for each scope are described below:
For CF-Q160L endoscope:
a. Bubbler flow rate: 4000 mL/min
b. Circulation Flow Rate: 12000 mL/min
c. Contact Time: 60 min

For XSIF-100 endoscope:
a. Bubbler flow rate: 4000 mL
b. Circulation Flow Rate: 11000 mL/min
c. Contact Time: 15 min and 30 min

For BF-P40 and BF-P30 endoscopes:
a. Bubbler flow rate: 4000 mL/min
b. Circulation Flow Rate: 11000 mL/min
c. Contact Time: 30 min

The chamber for all the runs was at room temperature.

TABLE A

| Endoscope Type | Result |
|---|---|
| Enteroscope | All 22 inoculated surfaces have no |
| (XSIF-100) | survivors |
| Colonoscope | All 22 inoculated surfaces have no |
| (CFQ-160L) | Survivors |
| Bronchoscope | All 22 inoculated surfaces have no |
| (BF-P40 and BF-P30) | Survivors |

All three types of flexible endoscope passed the 22 inoculated surfaces study.

### Example 2

Three runs were performed with the CFQ-160L scope described in Example 1 to determine if the efficacy is affected when approximately 100 CFU of bacteria were inoculated. The result showed that there were no survivors on all three runs which concluded that the efficacy of the system was not affected by the amount of bacteria present on the scope. One run of each with the BF-P40 and XSIF-100 was also performed to observe the effects of different scopes with 100 CFU inoculations. The result for the BF-P40 showed no survivors after 7 days.

The data show that there are no survivors at day 4. The results for the XSIF-100 with 100 CFU inoculation showed no survivors after 7 days.

Test conditions for all three runs were:
Bubbler flow rate: 4000 mL/min
Circulation Flow Rate: 12000 mL/min
Contact Time: 60 min
Chamber at room temperature.
Sterilant: Rapicide PA Part A
Bubbler used for Vapor Generator: 1L glass bubbler
Chamber size: 20 liter

### Example 3

A 60 liter stainless steel chamber was used to determine the effect of chamber size on the efficacy of the bubbler system when the chamber size and material were changed. The flow rate used was 4 L/min for a bubbler and approximately 12 L/min for circulation and the sterilization time was for 60 minutes. Twenty-two inoculated surfaces were studied with the colonoscope (CFQ-160 L). Each scope was inoculated on both the exterior and interior of the lumens with Geobacillus stearothermophilus suspended in serum or saline solution at a target concentration of microorganisms of 6-7 log for each run.

The results were that the 22 inoculated surfaces had no survivors for both scope and the biological indicators used, suggesting efficacy of sterilization was unaffected by a larger chamber size.

### Example 4

To evacuate the sterilant out after the sterilization process, the experiment utilized three additional parts to the system:
1. Circulating fan inside the chamber lid to generate turbulence to promote the evacuation process.
2. Air pump at the outlet vent to help remove the sterilant out of the system.
3. Medical air inlet to dilute the concentration of PAA for faster evacuating process.

The process included a flow of medical air in at 15 L/min (flow through both chamber and lumen of scope) and a flow of air out of the chamber (generated by the air pump) and a circulation flow inside the chamber (generated by the fan). The equivalent of flow through the chamber allowed the pressure inside the chamber to be closer to atmospheric pressure to reduce scope damage and allow easier design of the chamber. The results demonstrate that with a 30 minute ventilation time, the concentration of PAA inside the chamber was reduced to 160 parts per million, with residuals on the scope as low as 30 mg of PAA (well below the worst case residual toxicity of 7050 mg of PAA per total device).

### Example 5

Five experiments were performed using system 220. Detailed method parameters are given in the Table B. Infrared (IR) data was collected for the first manual Cycle and Cycle 223. The IR cell was operated at 40° C., averaging 6 scans at a resolution of 4 wavenumbers. IR data was not collected for all cycles due to the work required to obtain it and resources availability.

TABLE B
Cycle ID and Parameters for system 220

| | Contact | | | | | | | Post | | |
|---|---|---|---|---|---|---|---|---|---|---|
| | Time | Air | | Sterilant | | | Injection | Inj | Dehumid | Dehumid |
| Cycle #ID | (seconds) | Rate | Sterilant (v) | Weight | # Inj | Vents | torr | Torr | Torr | Hold |
| Manual 1 | 600 | NA | NA | NA | NA | NA | 20 | 20 | NA | NA |
| Manual 2 | 600 | NA | NA | NA | NA | NA | 20 | 20 | NA | NA |
| 223 | 3600 | 0 | 0 | 0 | 1 | 6 | 20 | 20 | 100 | 300 |
| 224 | 1800 | 0 | 0 | 0 | 1 | 10 | 20 | 20 | 100 | 300 |
| 225 | 1800 | 0 | 0 | 0 | 1 | 4 | 20 | 20 | 100 | 300 |

Referring to FIG. 7, a Mesa brand biological indicator (BI) and PAA chemical indicator (CI) were placed within a custom vessel 260 along the flow path of the bubbler system. The Mesa Biological indicators have a built in chemical indicator such that the chemical indicator is initially a red ring that turns white upon exposure to PAA vapor.

In each sample the CI at the distal tip showed PAA to be flowing through the elevator wire channel. However, Mesa biological indicators registered alive after 10 minutes of exposure time. Additionally, the chemical indicator on the Mesa BI did not register after 10 minutes of exposure to PAA. When scope flow time was increased to 30 minutes or greater, the biological indicators registered as dead and their associated chemical indicator had tripped.

TABLE C
Summarized results

| | Contact | | | | |
|---|---|---|---|---|---|
| | Time | | | CI | CI |
| Run ID | (seconds) | BI Results | CI (Vessel) | (Distal) | (Control) |
| Manual 1 | 600 | Alive/CI− | Negative | Positive | Negative |
| Manual 2 | 600 | Alive/CI− | Negative | Positive | N/A |
| 223 | 3600 | Dead/CI+ | Negative | Positive | Negative |
| 224 | 1800 | Dead/CI+ | Negative | Positive | N/A |
| 225 | 1800 | Dead/CI+ | Negative | Positive | Negative |

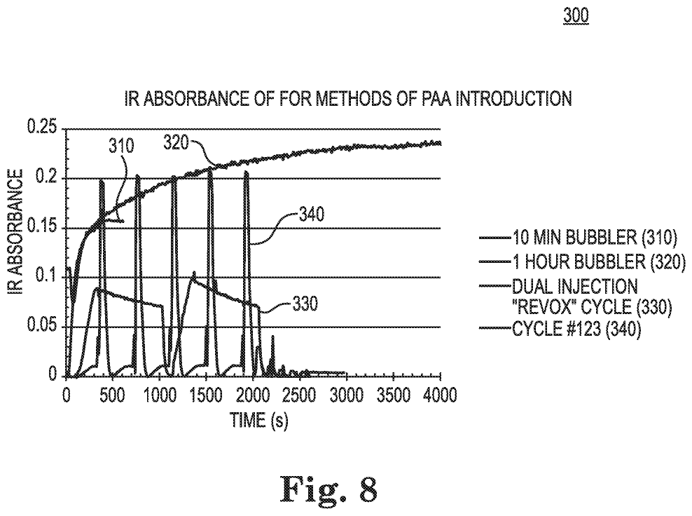

Graphs of the PAA levels are provided in FIG. 8. Data captured on graph 300 from infrared detector 254 illustrates a high PAA concentration at line 320 and line 310 of the IR signals for exposure times of a standard bubbler of 1 hour and 10 minutes, respectively. By comparison, line 330 shows a dual injection standard “REVOX cycle” and line 340 illustrates improved bubbler 230 (Cycle 123). Cycle 123 used the Agilent spray nozzle at an injection pressure of 10 torr with a total of 5 injections using 10 grams of REVOX PA per injection. This cycle currently stands as the highest PAA concentration created using air assisted spray. In each sample, the infrared detector was a part of the scope flow path. Dual injection REVOX cycle achieves peak IR signals around 0.9 absorption units, which is roughly 1% PAA per mol of total vapor. Cycle 123 (bubbler 230) achieved peak concentrations double that of a typical REVOX cycle injection. Bubbler 230 is able to sustain PAA concentrations exceeding that of even Cycle 123 for over an hour 320. Of note within these results is the fact chemical indicators within the CI/BI vessel never registered the presence of PAA, despite dead biological indicators, distal tip CI results, and the infrared data. This CrossTex CI is meant for the determination of PAA concentration in a liquid solution, not a gas phase PAA. The indicators turn from white to black after PAA exposure. Over exposure bleaches them back to white again. The BUCI vessel creates a large volume change to the gas as it flows. It is currently hypothesized the CI is the result of this volume change or is the result of a bleaching action. Later experiments showed the PAA chemical indicators can bleach back to white after turning black. The most likely scenario is that these strips bleached and appeared as though they were not activated.

### Example 6

In this example embodiment of bubbler 430 and system 420, the Bubbler System was modified to accept a 50 mm outside diameter by 90 cm long glass tube (5 mm wall thickness) attached to a 417-liter REVOX system. During all experiments, 250 mL of REVOX PA liquid was measured by graduated cylinder and poured into the 90 cm Bubbler. The Bubbler System was connected to the 417-liter REVOX sterilization chamber referred to as an improved bubbler vessel configuration. Air was drawn into bubbler 430 using only the force of the vacuum within sterilization chamber. A simple on-off valve was used to control the flow of air through the bubbler, allowing for “blocks” to be created in a similar manner to a normal REVOX cycle. In several cases a flow meter and/or infrared vapor detector was used to monitor the output of the Bubbler. In all cases, unused functions of the modified Revox Sterilization chamber/system were turned off (e.g. Dehumidification Hold was always set to 0) or were disabled (e.g. scope flow valve power was physically unplugged).

An assortment of process challenge devices (PCDs) were procured as a rough gauge to see what modes of sterilization are possible with the current bubbler 430 setup. These devices included simple Tyvek bags containing BIs, a syringe based PCD which contained a BI, a HF2000 Hemofilter cut to allow a BI to be inserted into the fiber mesh, and an artificial bone matrix provided by the REVOX team. Additionally, an Olympus TJF160 duodenoscope was inoculated with 6-log of bacteria on the exterior surface and within the scope lumens and was exposed to a Bubbler cycle with no hookups. This scope was used to assess penetration of sterilant down fine lumens.

### Example 7

### Non-Heated Bubbler Experiments and Results

Non-Heated Bubbler Experiments using the 90 cm bubbler 430 demonstrated no bacterial kill under a multitude of conditions. Shown in Table 1 are the results of 7 tests that attempted kill on simple EXPOSURE biological indicators (BIs). The BIs were not placed in any other packaging and were simply exposed to the chamber atmosphere during the sterilization run.

TABLE 1
Non-Heated Bubbler Runs and Results

| Cycle | Contact | | | | Inj | Post inj | Dehumid | BI | |
|---|---|---|---|---|---|---|---|---|---|
| Run ID | Time | Sterilant | # Inj | Vents | torr | torr | torr | Results | CI Results |
| 243 | 600 | 250 mL of | 3 | 4 | 10 | 500 | 10 | All | Positive; |
| | | chemistry | | | | | | positive; | Dark |
| | | | | | | | | CIs on | Black |
| | | | | | | | | the Mesa | |
| | | | | | | | | BIs had | |
| | | | | | | | | bleached | |
| | | | | | | | | out | |
| 244 | 600 | 250 mL of | 3 | 4 | 10 | 500 | 10 | All | Positive; |
| | | chemistry | | | | | | positive; | Dark |
| | | | | | | | | CIs on | Black |
| | | | | | | | | the Mesa | |
| | | | | | | | | BIs had | |
| | | | | | | | | bleached | |
| | | | | | | | | out | |
| 245 | 1200 | 250 mL of | 3 | 3 | 10 | 500 | 10 | Mesa All | Positive; |
| | | chemistry | | | | | | Positive, | Dark |
| | | | | | | | | CIs all | Black |
| | | | | | | | | negative | |
| 246 | 1800 | 250 mL of | 3 | 3 | 5 | 500 | 5 | Mesa All | Positive; |
| | | chemistry | | | | | | Positive, | Dark |
| | | | | | | | | CIs all | Black |
| | | | | | | | | negative | |
| 247 | 300 | 250 mL of | 20 | 3 | 5 | 500 | 5 | Mesa All | Positive; |
| | | chemistry | | | | | | Positive, | Dark |
| | | | | | | | | CIs all | Black |
| | | | | | | | | negative | |
| 248 | 1200 | 250 mL of | 3 | 3 | 5 | 500 | 5 | Mesa All | Positive; |
| | | chemistry, | | | | | | Positive, | CIs |
| | | Custom at | | | | | | CIs all | Bleached |
| | | 16.5% PAA | | | | | | negative | out |
| 249 | 120 | 250 mL of | 20 | 3 | 5 | 500 | 5 | Mesa All | Positive; |
| | | chemistry, | | | | | | Positive, | CIs |
| | | Custom at | | | | | | CIs all | Bleached |
| | | 16.5% PAA | | | | | | negative | out |

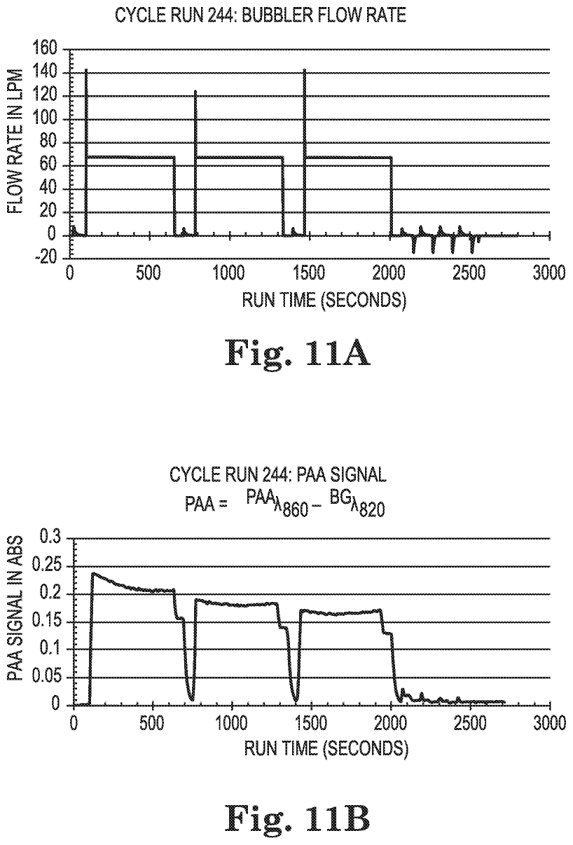

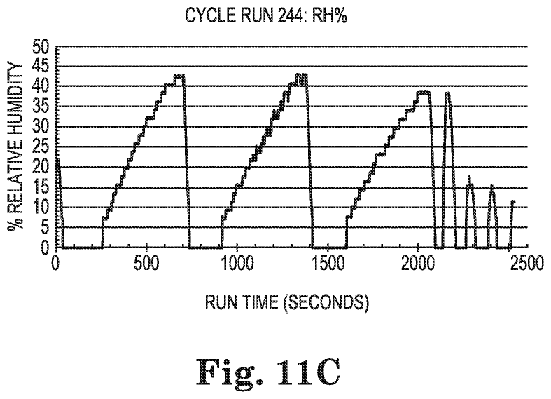

Referring to FIGS. 11A and 11B illustrate graphs of a bubbler flow rate, PAA signals for PAA introduction and relative humidity data from an oversized bubbler of FIG. 9B, respectively, all experiments which used the unheated Bubbler recorded no BI kill regardless of sterilization conditions. On the contrary, chemical indicator data demonstrated a very high PAA concentration within the sterilization chamber. In some cases, the PAA concentration was high enough to completely bleach out the chemical indicators after they had triggered (chemical indicators for PAA turn from white to black after PAA exposure; over exposure of PAA vapor turns them white again by a bleaching action). Infrared characterization and flow data registered from Cycle 244 again showed that a very high PAA concentration was present within the sterilization chamber.

To estimate the PAA content per liter of air, titration data was collected from Cycle 246. Simply put, the REVOX PA used for this experiment was titrated for PAA content before and after its use in the Bubbler. When combined with the flow meter data, it indicated that around 840 μg of PAA was being delivered per liter of air flowing through the Bubbler. This cycle did not give any kill but did confirm delivery of PAA exceeded what is considered a lethal dose for the BIs used in this experiment. Cycles 248 and 247 utilized a custom made PAA mixture at 16.5% PAA. When used in the unheated Bubbler System, this concentration was strong enough to completely bleach out the chemical indicator strips almost immediately upon exposure. Infrared and flow meter data were deliberately not collected for fear of damaging the instrumentation. It should also be noted, the PAA concentration was high enough to oxidize the glue on all the tape used to affix different apparatuses within the novel bubbler's chamber. The oxidation completely neutralized all adhesive properties of the tape, causing CIs to come lose and fall off the glass beakers. This phenomenon has not been recorded prior to, or since this work and is indicative of a high PAA concentration within the sterilization chamber. Titration of the PAA mix was attempted, but the disclosed methodology cannot account for the rapid reformation of PAA seen in this formulation and the data yielded no conclusive results. No microbiological kill was recorded for either of these cycles despite the PAA concentration.

A general reason for the lack of microbiological kill, even under extreme conditions, can be found in a 1968 publication by Dorothy Portner entitled Sporicidal Effect of Peracetic Acid Vapor (Portner). This paper reports a PAA delivery mechanism which yields PAA concentrations around 500 to 1500 μg of PAA per liter of air. This concentration range brackets the concentration seen using the disclosed Bubbler and standard REVOX PA chemistry, however all of the experiments were performed in ambient temperature and pressure and specifics were not provided on the equipment used. In the Portner paper, full kill of bacteria is not achieved unless the relative humidity of the system is greater than 60%. Using data and recorded data from Cycle 244 (see FIG. 11C), the relative humidity never exceeded 60% during the testing of the unheated bubbler. Therefore, a low humidity environment generated from the unheated bubbler merely confirms the findings of Portner.

Although this paper reports that “The corrosiveness of PAA has limited its practical application as a disinfectant”, PAA is not so corrosive as to be ineffective as is in the manner in which the paper formulated its PAA source. The paper reports the use of high levels of acetic acid catalyzed by sulfuric acid with the acetic acid and sulfuric acid being two corrosive species in this formulation. The paper further states that the “PAA itself is not adsorbed onto surfaces”, which does occur in example embodiments and at satisfactory levels. The PAA formulation of Portner appears to be as follows: the commercial grade of PAA solution is composed of approximately 40% PAA, 5% hydrogen peroxide, 39% acetic acid, 1% sulfuric acid, and 15%, water, w/w. This formulation has high levels of acetic acid in it and amounts of hydrogen peroxide, and uses sulfuric acid as a catalyst. This makes the Portner PAA solution fairly ineffective for vaporized sterilization due to the very high concentration of acetic acid and lower water content. Further, the Portner test chamber is very different from the various example embodiments taught herein in that Portner's chamber is not subjected to a vacuum and Portner's process for PAA introduction is different. Portner uses a mist or spray nozzle type apparatus. The spray system used by Portner will vaporize primarily acetic acid and PAA in the mixture. Because the spray system of Portner is operating at atmospheric pressure, the vaporization of hydrogen peroxide, sulfuric acid, and any other non-volatile components will be very, very minimal. Further, PAA needs water for its germicidal activity to be apparent (no water means no kill).

### Example 8

### Heated Bubble Experiments

Heated Bubbler experiments delivered greatly improved results of their non-heated predecessors. As shown in Table 2, Cycle 250 delivered complete kill on all BIs and CIs were bleached out completely in the exact same fashion as was observed in Cycles 248 and 249, which indicate a high PAA concentration.

TABLE 2
Heated Bubbler Experimental Conditions and BI/CI Results

| Cycle | Contact | # | # | Inj | Post Inj | Dehumid | BI | CI |
|---|---|---|---|---|---|---|---|---|
| Run ID | Time | Inj | Vents | Torr | Torr | Torr | Results | Results |
| 250 | 1200 | 3 | 3 | 5 | 500 | 5 | BIs in Beakers Negative | Positive: CI went black almost |
| | | | | | | | | immediately and then bleached out |
| | | | | | | | | over the course of the run. |
| 251 | 60 | 20 | 3 | 5 | 500 | 5 | BIs in Beakers Positive | Positive: CIs went very black and then |
| | | | | | | | | stayed black. Did not bleach out. |
| 252 | 1800 | 1 | 3 | 5 | 500 | 5 | BIs in Beakers Negative | Positive: CIs bleached out as per run |
| | | | | | | | | 250. |
| 253 | 1200 | 3 | 3 | 5 | 500 | 5 | BIs in Beakers: Negative | Positive: CIs turned black and then |
| | | | | | | | BIs in Tyvek: Negative | bleached out to white after |
| | | | | | | | Scope Exterior: Full Kill | approximately 5 minutes of exposure. |
| | | | | | | | Scope Lumens: ~1 log reduction | |
| 254 | 900 | 1 | 3 | 5 | 500 | 5 | BIs in Beakers: Negative | Positive: Both the 1700 ppm and trace |
| | | | | | | | BIs in Tyvek: Negative | level CIs were used. Both turned black |
| | | | | | | | | and then bleached out at the same |
| | | | | | | | | rate. |
| 255 | 1200 | 3 | 3 | 5 | 500 | 5 | BIs in Beakers: Negative | Positive: Turned black and then |
| | | | | | | | BIs in Syringe PCD: Negative | bleached out. |
| | | | | | | | Bone Marrow BI: Positive | |
| | | | | | | | Hemofilter PCD BI: Positive | |
| 256 | 3600 | 2 | 3 | 1 | 500 | 1 | BIs in Beakers: Negative | Positive: Turned black and then |
| | | | | | | | Syringe PCD: Negative | bleached out. |
| | | | | | | | Bone Marrow PCD: Positive | |
| | | | | | | | Hemofilter PCD: Positive | |

The difference between heated and non-heated bubbler systems is the resulting humidity present in the chamber during the sterilization cycles. The humidity level on Cycle 250, as an example, exceeded 100% relative humidity. Maximum humidity levels were recorded by visual observation. The importance of humidity was again shown in Cycle 251 where humidity levels never exceeded 60% while testing a rapid, multi block cycle. No kill on BIs was observed in Cycle 251. Cycle 252 was used to test the effect of a single block run with a longer period of hold. Kill was achieved during the cycle and humidity levels again exceeded 100%. Cycle 253 tested the penetration of Bubbler created PAA vapor into the long lumens of an Olympus TJF-160 duodenoscope. The scope surface, elevator wire channel, air/water channel, and suction biopsy channel were each inoculated with 6-logs of Geobacillus stearothermophilus spores. No hookups were used to pass PAA vapor into the lumens. For this experiment, a full kill of the spores was only observed on the surface of the scope and a 1-log reduction was observed in the lumens. This suggests hookups and a mechanical system to move PAA vapor are needed to achieve kill within endoscope lumens on a reasonable time scale using the Bubbler vaporization technology.

Cycle 254 tested a single 15-minute contact time on BIs in the chamber and within a Tyvek pouch. With pump down and ventilation, the total run time was 22 minutes. All the BIs were found to be sterilized. Additionally, between 5 and 10 additional minutes could be cut out of the cycle to achieve the same results. This is based on the rate at which the chemical indicators bleach out within the chamber, which tends to happen less than 5 minutes after PAA exposure begins. Within this cycle as well, two different types of Cantel Medical PAA chemical indicators were used. No appreciable difference was observed between the two varieties of CIs. Cycles 255 and 256 provide insight into the utility of the large scale Bubbler technology. While the PAA vapor generated from the Bubbler was able to kill the BI within the syringe PCD, it was not able to kill the BI within the bone mesh PCD or the MarCor Hemofilter. The results of the Hemofilter can be explained by the complexity of filter membrane and its massive surface area. More “blocks” or injections of PAA vapor are likely needed to saturate the filter membrane with PAA vapor. The bone matrix, however, is readily porous and should be easily sterilized under these conditions. XRF analysis of the bone matrix revealed high concentrations of metal ions including magnesium and calcium. It is hypothesized that the metal is causing a breakdown of the PAA vapor as it passes through the bone matrix.

In another experiment using a the bubbler having the 50 mm outside diameter by 90 cm long glass heated with a 6.1 meter (20 feet) long silicone rubber heating tape described above, the vapor PAA concentration in the chamber was determined by measuring the PAA concentration removed from the chamber containing vaporized PAA using FTIR (NICOLET iS5 spectrophotometer) Before the PAA concentration was determined, 250 mL Rapicide PA Part A (equivalent to REVOX PA chemistry) was added to the bubbler and the bubbler was heated to about 50° C. (about 120° F.). The vapor PAA concentration was determined as follows:

Start each cycle by pulling down the chamber down to 10 torr. Then the bubbler was allowed to run at maximum flow rate with the current setup (valve fully opened) to fill the chamber with the vapored sterilant until the chamber pressure reached 500 torr. At 500 torr, the bubbler was turned off and the chamber was allowed to equilibrate for 10-15 seconds. After the equilibration time passed, the vacuum through the FTIR system was turned on to pull the vapor inside the chamber through the FTIR system. The sampling process was run for 2 minutes. After the sampling process completed, the system was vented through 4 vent cycles to evacuate all remaining sterilant inside the chamber. The sampling was repeated three times using the same chemistry. The results are listed in Table 3. The results showed that the vapor concentrations generated by the bubbler system (with the vaporization phase from 10 torr to 500 torr) ranged between 4.80 mg/L to 5.83 mg/L PAA.

TABLE 3
Vapor concentration results for bubbler system with 120 L test bed.
Vapor PAA Concentration with Bubbler System

| Run # | Max IR Abs | PAA Vapor (mg/L) |
|---|---|---|
| 1 | 0.426352 | 5.83 |
| 2 | 0.383755 | 5.25 |
| 3 | 0.350746 | 4.80 |

This invention has been described above in terms of specific embodiments, but it is to be understood that the invention is not limited to these disclosed embodiments. Upon reading this disclosure many modifications and other embodiments of the invention will come to mind of those skilled in the art to which this invention pertains, and which are intended to be and are covered by both this disclosure and the appended claims. It is intended that the scope of the invention should be determined by proper interpretation and construction of the appended claims and their legal equivalents, as understood by those of skill in the art relying upon the disclosure in this specification and the attached drawings.

What is claimed is:

1-21. (canceled)

22. A method of sterilizing a medical device comprising:
bubbling a gas through an aqueous peracetic acid solution at a temperature less than about 60° C. to generate peracetic acid vapor;
delivering the peracetic acid vapor to a sterilization chamber containing a medical device needing to be sterilized, wherein the sterilization chamber is at a temperature of about 18° C. to 35° C.;
contacting the outer surfaces and inner surfaces of the medical device with the peracetic acid vapor;
recycling the peracetic acid vapor in the sterilization chamber until the medical device is sterilized; and
removing the peracetic acid from the sterilization chamber by applying a vacuum source to the sterilization chamber to provide the sterilized medical device.

23. The method of claim 22 further comprising the step of neutralizing the peracetic acid when it is removed from the sterilization chamber.

24. The method of claim 22, wherein the step of bubbling the gas through an aqueous peracetic acid is at a rate of about 1000-5000 ml/minute.

25. The method of claim 22, wherein the step of delivering the peracetic acid vapor to the sterilization chamber is at a rate of about 2000-12000 ml/minute.

26. The method of claim 22, wherein the step of contacting the medical device with the peracetic acid is for about 15-60 minutes.

27. The method of claim 22, wherein the step of contacting the inner surface of the medical device is by adjusting flow rate through channels of the endoscope at least about 500 ml/minute.

28. The method of claim 22, wherein the step of recycling the peracetic acid is at a rate of about 10000-14000 ml/minute.

29. The method of claim 22, wherein the medical device is an endoscope.

30. The method of claim 22, wherein the peracetic acid vapor generator is a sparger.

31. The method of claim 22, wherein the gas is air, argon, or nitrogen.

32. The method of claim 22, wherein the contacting the medical device is with a peracetic acid concentration of about 500-2500 ppm, a hydrogen peroxide concentration of less than about 100 ppm and an acid concentration of less than about 100 ppm.

33. The method of 22, wherein the aqueous peracetic acid solution is heated to a temperature in the range from about 40° C. to about 60° C. before the step of delivering the peracetic acid.

34. The method of claim 22, wherein the sterilization chamber is substantially free of internal corners or edges,

35. The method of claim 34, wherein the sterilization chamber is substantially ellipsoid, ovoid or spheroid.

36-42. (canceled)

43. A peracetic acid generator device comprising:
a peracetic acid (peracetic acid) bubbler device containing an aqueous peracetic acid solution, the peracetic acid bubbler device having an elongate container configuration with an elongated inlet tube located longitudinally therein with a distal portion of the inlet tube disposed adjacent a floor of the elongate container and a proximal portion of the inlet tube protruding from a top of the elongate container and open to a source of gas, the peracetic acid bubbler device having an outlet tube disposed through the top with a distal portion of the outlet tube located within the elongate container in an open space above a maximum level of the peracetic acid solution and with a proximal portion of the outlet tube providing an outflow path for a peracetic acid gas;
wherein the peracetic acid bubbler device containing the maximum level of the peracetic acid solution is adapted to be partially subjected to a vacuum environment and the proximal portion of the inlet tube and the proximal portion of the outlet tube extend outside of the vacuum environment, an outflow of a peracetic acid gas through the outlet tube being controllable by the vacuum environment at a flow rate defined by a height and volume of the elongate container and a length of the inlet tube.

44. The device of claim 43, wherein the elongate container is comprised of an elongate sealed glass tube having an outer diameter of about 50 mm and a length of about 90 mm capable of containing about 250 mL of a sterilant solution and an elongated inlet tube made of stainless steel and having a diameter of about 0.25 inches.

45. A method of sterilizing a medical device or appliance comprising:
providing a peracetic acid bubbler device containing an aqueous peracetic acid inlet tube located longitudinally therein with a distal portion of the inlet tube disposed adjacent a floor of the elongate container and a proximal portion of the inlet tube protruding from a top of the elongate container and open to a source of gas, the peracetic acid bubbler device having an outlet tube disposed through the top with a distal portion of the outlet tube located within the elongate container in an open space above a maximum level of the peracetic acid solution and with a proximal portion of the outlet tube providing an outflow path for a peracetic acid gas;
subjecting the peracetic acid bubbler device containing the peracetic acid solution to a vacuum environment such that the proximal portion of the inlet tube and the proximal portion of the outlet tube extend outside of the vacuum environment; and
bubbling the source gas through the peracetic acid solution from the inlet tube initiated by the vacuum environment and generating an outflow of a peracetic acid vapor gas through the outlet tube, wherein the elongate container is of a defined height and volume and the elongated inlet tube is of a defined length to facilitate an inflow of a gas through the inlet tube at a defined flow rate.

46. The method of claim 45, wherein the step of subjecting the peracetic acid bubbler device includes the step of providing a vacuum of to a range of 0.5 torr to 500 torr.

47. The method of claim 45, wherein the medical appliance is an endoscope and further including the step of connecting the outlet tube of the peracetic acid bubbler device to an inlet of the endoscope for sterilizing an elevator wire channel.

48. The method of claim 47, further including the step of subjecting the endoscope to a peracetic acid vapor gas flow for a time period of about 10-60 minutes.
